(19) United States
(12) Patent Application Publication
(10) Pub. No.: US 2010/0016812 A1
(43) Pub. Date: Jan. 21, 2010

(54) Medical laser gel applicator apparatus and system

(75) Inventors: Jeff Latimer, Camas, WA (US); Joseph Hart, Camas, WA (US)

Correspondence Address: RYLANDER & ASSOCIATES PC, 406 West 12th Street, Vancouver, WA, 98660, US

(73) Assignee: Medical Discoveries, Inc.

(21) Appl. No.: 12/218,786

(22) Filed: Jul. 18, 2008

(51) Int. Cl. A61M 35/00

(52) U.S. Cl. 604/289

(57) ABSTRACT

A medical laser gel applicator apparatus for applying surgical gel for craniofacial surgery includes a gel tube having an application tip from which gel is applied to a patient, and a bottom slidably movable within said tube pushing gel out through said application tip, a gun having a handle, a chamber into which said gel tube inserts, a plunger with a shaft and a plunger face in which said plunger face contacts said removable bottom, a trigger in electromechanical communication with said plunger wherein when said trigger is depressed the plunger pushes against said bottom sliding said bottom in said tube, pressuring said gel out of said application tip, and wherein when said trigger is released, said plunger backs off of said bottom, stopping the flow of said gel.

### FIELD OF THE INVENTION

The present invention relates to apparatus and systems related to application of surgical gel to a patient.

### BACKGROUND

Medical laser craniofacial surgery utilizes a surgical gel to prepare the patient for the surgical operation. Application of the gel typically is provided in tubes that must be hand squeezed, with resulting mess on the hands, and overflow of the squeezed gel. Currently no system or apparatus is known for applying surgical gel to a patient using an electromotor operated apparatus in which it is easy to replace application tubes, which provides for an automatic reverse at the end of an application to stop overflow.

Presently known art attempts to address this problem, but has not completely solved the problem. The following represents a list of known related art:

| | Applicant/Issued | Date of |
|---|---|---|
| Reference: | to: | Issue/Publication: |
| US Pat Pub. 2003/0165793 A1 | Yobel et al. | Sep. 4, 2003 |
| U.S. Pat. No. 6,929,157 | Orecchia et al. | Aug. 16, 2005 |
| U.S. Pat. No. 6,095,814 | Petrich et al. | Aug. 1, 2000 |
| U.S. Pat. No. 6047,864 | Winkler | Apr. 11, 2000 |
| U.S. Pat. No. 5,749,968 | Melanson et al. | May 12, 1998 |
| U.S. Pat. No. 4,994,065 | Gibbs et al. | Feb. 19, 1991 |
| U.S. Pat. No. 4,768,954 | Dragan | Sep. 6, 1988 |
| U.S. Pat. No. 4,569,662 | Dragan | Feb. 11, 1986 |
| U.S. Pat. No. 4,425,121 | Young et al. | Jan. 10, 1984 |
| U.S. Pat. No. 4,406,654 | Bristow | Sep. 27, 1983 |

The teachings of each of the above-listed citations (which does not itself incorporate essential material by reference) are herein incorporated by reference. None of the above inventions and patents, taken either singularly or in combination, is seen to describe the instant invention as claimed.

United States Patent Application Publication No. 20030165793 to Yobel, et al, published Sep. 4, 2003 for a “System for dispensing viscous materials”, discloses an invention which provides for a delivery system where a viscous material such as dental composite is heated by an induction field or by resistance to an electric current. The system comprises at the proximal end a capsule-like cartridge that is self heating. The capsule may be fabricated of any number of heat conducting polymers or doped polymers that are susceptible to induction fields or any material that will heat when an induction current is applied. In the second instance the capsules may have a resistance wire of the appropriate metal or any other material to allow heating of the capsule when a current is applied. In the third instance the capsule may have an induction coil embedded in it's wall combined with any type of metal or other material where this becomes the heating device when current of the appropriate nature is applied. In the fourth instance a thin film or foil may be applied to either the outer surface or the inner surface of the capsule such that when a current is applied the foil or film heats and in so doing heats the capsule and the contents. Coupled with the above described capsule is a delivery device. This hand held device, whether powered manually or electrically forces a shaft to engage a piston embedded within the cartridge to move forward, dispensing the material within the cartridge at it's proximal end. In the manually powered mode, an electrical power source, whether a battery, capacitor discharge or AC/DC current, is used exclusively to activate the various heating methods described above. In the electrically powered mode, a linear stepper motor or other such motor with a proper configuration allows the shaft to engage the piston embedded in the cartridge and allow the material contained within the cartridge to be dispensed at it's proximal end. In the electrically powered configuration the power source described above is used to power the motor as well as the energy to activate the various heating elements described above. In addition where the force needed to extrude-the material contained in the capsule, embedded in the device, whether manually or electrically applied to the device, such device may have the induction coil embedded in the barrel extension of the device in such placement as to provide adequate heating of the capsule.

U.S. Pat. No. 6,929,157 issued Aug. 16, 2005 to Orecchia, et al., for a “Multiple use dental viscous material dispenser” discloses a viscous material dispenser, and dispenser system and kit, including a dispenser without a dispensing tip and one or a plurality of separate dispensing tips that are removably attachable to the dispenser. The dispenser can be used with many different types of viscous material containing capsules and cartridges, such as those containing composites, dental cements, glass ionomers and the like. In embodiments, the dispenser can incorporate an integral activator for multi-component composition cartridges, such as glass ionomer cartridges.

U.S. Pat. No. 6,095,814 to Petrich, et al. issued Aug. 1, 2000 for “Dispensing cartridge with stepped chamber” discloses a dispensing cartridge for use with a hand-held applicator having a chamber with a first section and a second section. The second section has a smaller cross-sectional area than the first section such that the chamber has a stepped configuration. A piston is slidably received in the chamber and has a head portion that is received in the second section and a tail portion that is received in the first section. As a force is applied to the tail portion, the head portion advances to expel material through an outlet opening. The cartridge is useful for dispensing compositions having a relatively high viscosity such as dental pastes and the like.

U.S. Pat. No. 6,047,864 to Winkler issued Apr. 11, 2000 for “Actuating device” discloses an actuating device for dispensing of a paste-like material, especially in the dental field, from a capsule or cartridge having an abutment for the support of the capsule or cartridge at the actuating device in the direction of dispensing. The abutment is provided at a collar-shaped projection and is a part of at least one securing tongue. The securing tongues are provided at the housing of the actuating device and can be released for releasing the capsule or cartridge.

U.S. Pat. No. 5,749,968 to Melanson, et al., issued May 12, 1998 for a “Device for priming for improved adherence of gels to substrates” discloses an improved barrier or drug delivery system which is highly adherent to the surface to which it is applied, along with methods for making the barrier. In the preferred embodiment, tissue is stained with a photoinitiator, then the polymer solution or gel having added thereto a defined amount of the same or a different photoinitator is applied to the tissue. On exposure to light, the resulting system polymerizes at the surface, giving excellent adherence, and also forms a gel in the rest of the applied volume. Thus a gel barrier of arbitrary thickness can be applied to a surface while maintaining high adherence at the interface. This process is referred to herein as “priming”. The polymerizable barrier materials are highly useful for sealing tissue surfaces and junctions against leaks of fluids. In another embodiment, “priming” can be used to reliably adhere preformed barriers to tissue or other surfaces, or to adhere tissue surfaces to each other. A first surface and a barrier, or another surface, are prestained with initiator, and a thin layer of gellable monomer containing initiator is placed between them. Strong adhesion is obtained between the two surfaces on gelation of the monomer. In a similar fashion, tissue surfaces can be adhered to each other in repair of wounds and formation of anastomoses. Methods for use of non-photochemical systems and combined chemical/photochemical systems are described.

U.S. Pat. No. 4,994,065 to Gibbs, et al. issued Feb. 19, 1991 for an “Apparatus for dispensing low viscosity semi-fluid material under pressure” discloses an injection gun especially useful for injecting low volume, high pressure shots of bone cement. The gun includes a cylindrical housing with a plunger including a plunger rod extending axially through the housing. The plunger rod includes a segment which is provided with an axial series of notches. A ratchet block is slidable axially within the cylindrical housing and is concentric with the plunger rod. The ratchet cylinder mounts a ratchet pawl urged against the plunger rod and adapted to engage and lock into the series of notches for forward movement. A pistol grip extends from the cylindrical housing, and a lever is pivoted thereto with an extension of the lever engaging the ratchet cylinder for advancing the plunger. A second ratchet pawl is mounted on a fixed member of the cylindrical housing for retaining the plunger against rearward movement when the pawl in the ratchet cylinder is disengaged from the rod.

U.S. Pat. No. 4,768,954 to Dragan issued Sep. 6, 1988 for a “Syringe tip” discloses a dispensing nozzle tip for use with dental syringe which is adapted to be detachably connected to the end of a dental syringe barrel having a uniquely shaped dispensing nozzle which enables the extruded dental material to be dispensed as a flat ribbon for direct placement onto a patient's tooth in a manner which minimizes the time and effort on the part of a dentist to coat a tooth with such dental material; and which nozzle tip is rendered readily disposable to prevent cross-contamination when the dental syringe is used on successive patients.

U.S. Pat. No. 4,569,662 to Dragan issued Feb. 11, 1986 for a “Bulk cartridge for packaging and dispensing a dental material” discloses a dental syringe for the dispensing of a relative viscous dental material that includes a readily replaceable barrel portion that defines a reservoir for containing a bulk supply of a dental material having a spout or nozzle discharge end to which a dispensibly nozzle tip is detachably connected and which barrel portion is sealed by a displaceable plug. A plunger is provided for effecting the displacement of the plug for effecting the extruding of the material from the barrel portion as needed, and which plunger has formed on the end thereof a unidirectional vent arrangement which is operative for imparting a negative pressure on the end plug to permit a slight rearward displacement thereof to relieve the back pressure exerted on the plug by the material upon the termination of an extruding operation and thereby prevent drooling of any unwanted material subsequent to an extruding operation.

U.S. Pat. No. 4,425,121 to Young, et al. issued Jan. 10, 1984 for an “Adjustable feeding device for the administration of dosages of gels and pastes to farm animals” discloses an animal feeding device which accepts cartridges containing various gels, pastes, or other similar materials. This device is useful for the oral administration of feeds or medicants to farm animals, especially cattle, in adjustable dosages to fit the individualized needs of animals under treatment.

U.S. Pat. No. 4,406,654 to Bristow issued Sep. 27, 1983 for an “Adjustable feeding device for the administration of dosages of gels and pastes to farm animals” discloses an animal-feeding device which accepts cartridges containing various gels, pastes, or other similar materials. This device is useful for the oral administration of feeds or medicants to farm animals, especially cattle, in adjustable dosages to fit the individualized needs of animals under treatment.

Still other features would be desirable in a surgical gel applicator. For example, an applicator allowing easy replacement out of applicator tubes, elimination, of the overflow application problem, and an automatic reverse at the end of a gel application which stops gel flow.

Thus, while the foregoing body of art indicates it to be known to have a medical paste applicator, the art described above does not teach or suggest a surgical gel applicator apparatus and system which has the following combination of desirable features: (1) a surgical gel applicator for applying surgical gel to a patient that allows for easy replacement of applicator tubes, which can be refillable or disposable; (2) minimizes or eliminates the overflow application problem; and (3) an automatic reverse at the end of a gel application which stops gel flow, among others.

### SUMMARY AND ADVANTAGES

A medical laser gel applicator apparatus for applying surgical gel for cranial-facial surgery includes a gel tube having an application tip from which gel is applied to a patient, and a removable bottom slidably movable within said tube pushing gel out through said application tip, and which removes to allow refilling said gel tube, a gun having a handle, a chamber into which said gel tube inserts, a plunger with a shaft and a plunger face in which said plunger face contacts said removable bottom, a trigger in electro mechanical communication with said plunger wherein when said trigger is depressed the plunger pushes against said bottom sliding said bottom in said tube, pressuring said gel out of said application tip, and wherein when said trigger is released, said plunger backs off of said bottom, stopping the flow of said gel. Gel applicator can include a stepper motor with a controller, and a cog wheel engaging teeth on said plunger shaft.

The gel applicator apparatus and system of the present invention presents numerous advantages, including: (1) a surgical gel applicator for applying surgical gel to a patient that allows for easy replacement of applicator tubes, which can be disposable or refillable; (2) minimizes or eliminates the overflow application problem; and (3) an automatic reverse at the end of a gel application which stops gel flow, among others. Additional advantages of the invention will be set forth in part in the description which follows, and in part will be obvious from the description, or may be learned by practice of the invention. The advantages of the invention may be realized and attained by means of the instrumentalities and combinations particularly pointed out in the appended claims. Further benefits and advantages of the embodiments of the invention will become apparent from consideration of the following detailed description given with reference to the accompanying drawings, which specify and show preferred embodiments of the present invention.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

FIG. 1 shows an embodiment of a medical laser gel applicator apparatus and system.

FIG. 2 shows another view of the embodiment of a medical laser gel applicator apparatus and system shown in FIG. 1.

FIG. 3 shows an embodiment of a gel tube for a medical laser gel applicator apparatus and system.

FIG. 4 shows another view of the gel tube of FIG. 3.

FIG. 4A shows another view of the gel tube of FIG. 3.

FIG. 5 shows a front view of the embodiment of a medical laser gel applicator apparatus and system shown in FIG. 1.

FIG. 6 shows a rear view of the embodiment of a medical laser gel applicator apparatus and system shown in FIG. 1.

FIG. 7 shows a right side view of the embodiment of a medical laser gel applicator apparatus and system shown in FIG. 1.

FIG. 8 shows a left side view of the embodiment of a medical laser gel applicator apparatus and system shown in FIG. 1.

FIG. 9 shows a top down view of the embodiment of a medical laser gel applicator apparatus and system shown in FIG. 1.

FIG. 10 shows a bottom up view of the embodiment of a medical laser gel applicator apparatus and system shown in FIG. 1.

FIG. 11 shows another view of the embodiment of a medical laser gel applicator apparatus and system shown in FIG. 1 in use.

FIG. 12 shows a view of the internal operation of an embodiment of a medical laser gel applicator apparatus and system.

FIG. 13 shows another view of the embodiment of a medical laser gel applicator apparatus and system shown in FIG. 1.

FIG. 14 shows another view of the embodiment of a medical laser gel applicator apparatus and system shown in FIG. 1.

FIG. 15 shows a view of an embodiment of the cogwheels and worm gears of a medical laser gel applicator apparatus and system shown in FIG. 1.

### REFERENCE NUMBERS USED IN DRAWINGS

The following reference numbers are used in the drawings.

10 Medical laser gun gel applicator apparatus

12 Gel tube

14 Application tip

15 Gel tube base

16 Bottom

18 Gun housing

20 Handle

22 Chamber

23 Chamber lid

24 Trigger

26 Reverse switch

28A Gel tube front holder top

28B Gel tube front holder bottom

30 Plunger

30A Plunger face

30B Plunger shaft

30C Plunger shaft teeth

32A Stepper motor

32B Stepper motor controller

34a-i Cogwheels and worm gears

36 Battery holder

G Gel

P Patient

### DETAILED DESCRIPTION

Before beginning a detailed description of the subject invention, mention of the following is in order. When appropriate, like reference materials and characters are used to designate identical, corresponding, or similar components in differing figure drawings. The figure drawings associated with this disclosure typically are not drawn with dimensional accuracy to scale, i.e., such drawings have been drafted with a focus on clarity of viewing and understanding rather than dimensional accuracy.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

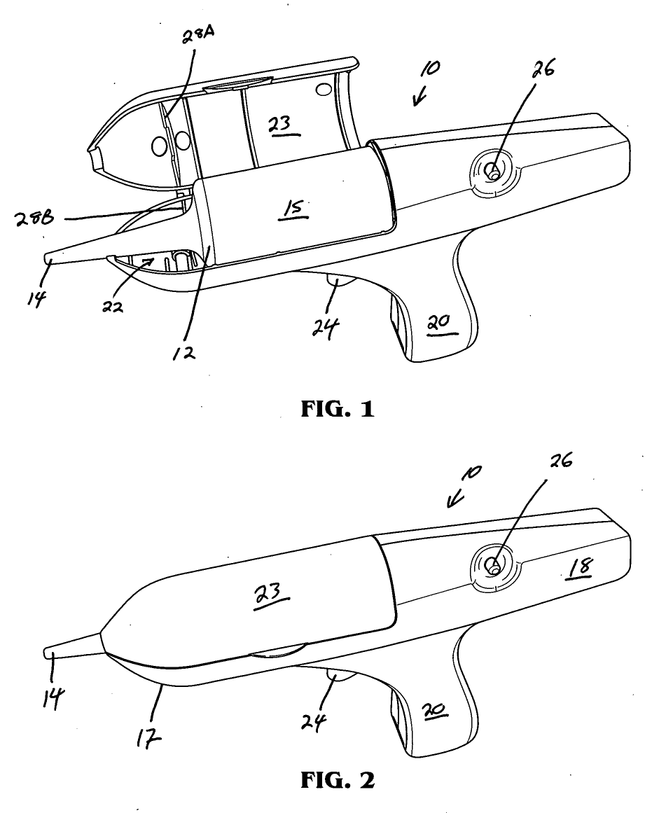

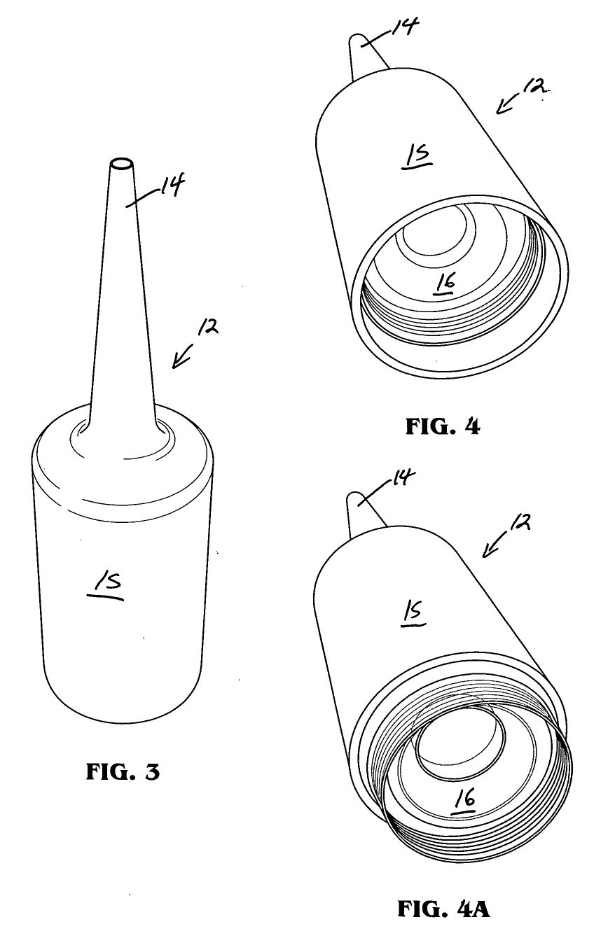

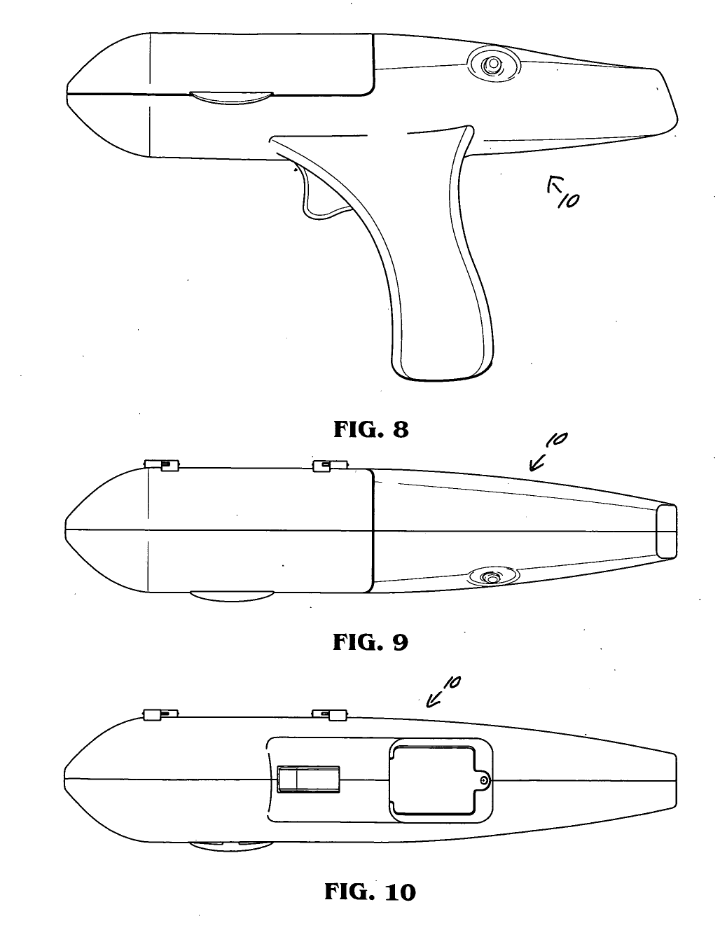

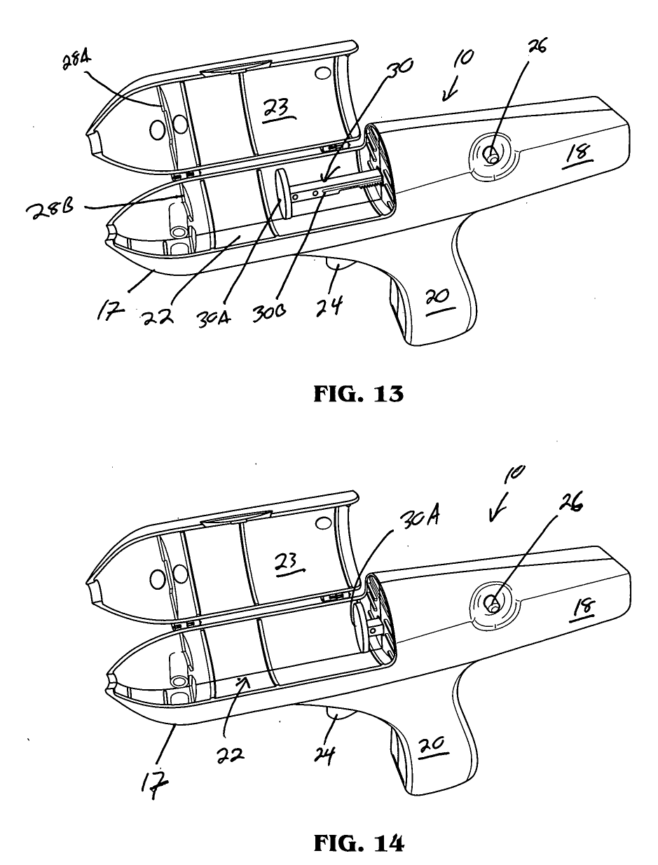

As shown in FIGS. 1-15, a medical laser gel applicator apparatus 10 is provided and includes a gel tube 12 having a application tip 14 from which gel G is applied to a patient P, and a bottom 16 slidably movable within said tube 12 pushing gel G out through said application tip 14, a gun 17 having a handle 20, a chamber 22 into which said gel tube 12 inserts, a plunger 30 having a shaft 30B and a face 30A wherein the face 30A contacts said bottom 16, a trigger 24 in electro mechanical communication with said plunger 30 wherein when said trigger 24 is depressed the plunger 30 pushes against said bottom 16 sliding said bottom 16 in said tube 12, pressuring said gel G out of said application tip 14, and wherein when said trigger 24 is released, said plunger 30 backs off of said bottom 16, stopping the flow of said gel G through pressure, and a motor for moving said plunger 30 in response to said trigger 24.

Gel applicator tube 12 is preferably a hollow columnar tube having a narrowed application tip 14 and a base 15. A bottom 16 on said tube 12 slides movably within the gel applicator tube 12, reachable from the base 15. The tube can be disposable. The bottom 16 can be removable so that the gel applicator tube 12 can be refilled with gel G.

The medical laser gel applicator apparatus 10 has a handle 20, a plunger housing 18, a tube chamber 22, trigger 24, and a plunger 30. Plunger 30 extends into the chamber 22 and has a plunger face 30A connected to the end of a plunger shaft 30B. Plunger 30 extends into the housing 18 and preferably includes teeth 30C along the shaft 30B. Housing 18 houses the motor for moving said plunger 30 in response to said trigger 24.

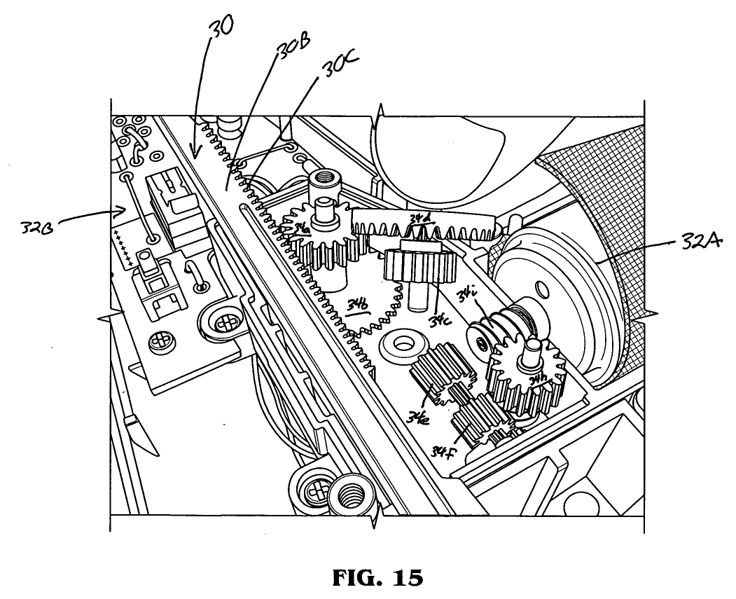

The motor can be a stepper motor 32A with a controller 32B, and one or more worm gears and cogwheels 34a-i in electromechanical communication through said controller 32B and motor 32A with said trigger 24 engaging the teeth 30C of said plunger shaft 30B. Stepper motor 32A and controller 32B drives the plunger 30 in response to the trigger 24 by rotating worm gears and cog wheels 34a-i one of which engages the teeth 30C on the plunger shaft 30B. See FIGS. 12 and 15. When the trigger 24 is depressed, the controller 32B causes the stepper motor 32A to move the plunger 30 toward the application end of the tube chamber 22, bringing the plunger face 30A into contact with the bottom 16, and thereby pushing the bottom 16 into the gel tube 12, pressurizing the gel G which is dispensed through the application tip 14. Gel tube 12 is stopped from moving forward by the gel tube front holder top 28A and bottom 28B which, when the chamber lid 23 is closed, create a holder for the front of the gel tube 12. When the trigger 24 is released, the stepper motor 32A automatically rotates in reverse for several rotations, thereby pulling the plunger 30 back, removing pressure from the gel tube 12. The pressure in the gel tube 12 forces the removable bottom 16 backward due to the high flow resistance of the tip 14 compared to the bottom 16, thereby causing any excess gel G at the tip 14 to be drawn back into the gel tube 12. The medical laser gel applicator apparatus 10 is preferably provided with a reverse switch 26 which, when depressed, causes the stepper motor 32A to draw the plunger 30 back. Those of ordinary skill in the art will know that stepper motors, controllers therefore and cogwheels to move a shaft forwardly and rearwardly are well known in the art and can be figured in numerous ways without undue experimentation.

Preferably, the handle 20 is hollow and contains a battery holder 36 to one or more batteries to power the stepper motor 32A and controller 32B.

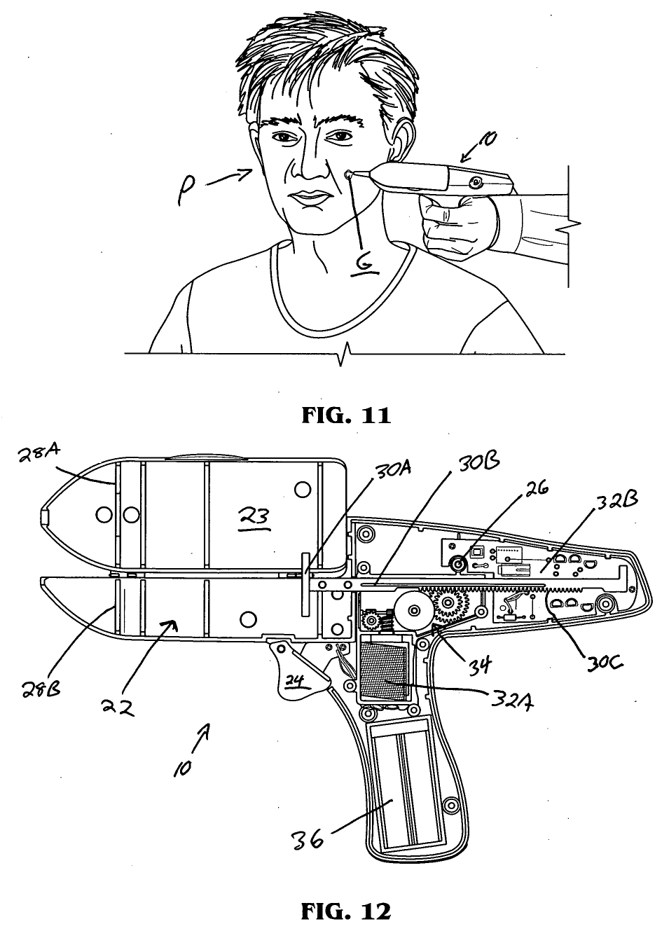

In operation in one embodiment, the gel applicator gun gel tube chamber lid 23 is opened. A full gel tube 12 is inserted the in the chamber 24, with the base 15 in proximity to the plunger face 30A and the application tip 14 extending out the front of the medical laser gun gel applicator apparatus 10. The chamber lid 23 is closed. The application tip 14 is put into proximity to the face of a patient P, FIG. 11. The trigger 24 is depressed. The plunger 30 extends into the chamber 22 coming into contact with the gel application tube bottom 16, and pushes the bottom 16 toward the application tip 14, pushing applicator gel G out the application tip 14 onto face of the patient P, FIG. 11. When the application is completed, the trigger 24 is released. The plunger 30 backs up, stopping the flow of the application gel G. In one embodiment, the gel tube 12 is disposable and replaced with a gel filled gel tube 12. Alternatively, the gel tube 12 is refillable with the bottom 16 removable. In this alternative, to refill or replace the application gel tube 12, the reversal switch 26 is applied to return the plunger 30 to the starting position, the chamber lid 23 is opened, the tube 12 removed, the removable bottom 16 removed, and gel G filled into the now empty application gel tube 12. The bottom 16 is reinserted. The chamber lid 23 is closed, and the apparatus 10 is ready to go again.

Those skilled in the art will recognize that numerous modifications and changes may be made to the preferred embodiment without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the preferred embodiment is essential. Other embodiments are possible, their specific designs depending upon the particular application. As such, the scope of the invention should not be limited by the particular embodiments herein described but should be defined only by the appended claims and equivalents thereof.

I claim:

1. A medical laser gel applicator apparatus, comprising:
a gel tube having an application tip from which gel is applied to a patient, and a bottom slidably movable within said tube pushing gel out through said application tip;
a gun having a handle, a chamber into which said gel tube inserts, a plunger with a shaft and a plunger face contactable with said removable bottom, a trigger in electromechanical communication with said plunger, wherein when said trigger is depressed the plunger pushes against said bottom sliding said bottom in said tube, pressuring said gel out of said application tip, and wherein when said trigger is released, said plunger backs off of said bottom, stopping the flow of said gel; and
electromechanical means for moving said plunger in response to said trigger depressing and releasing.

2. The gel gun of claim 1, wherein said electromechanical means include a stepper motor, a controller in electrical communication with said stepper motor, and gear in mechanical communication with said motor engaging teeth on said plunger shaft.

3. The gel applicator of claims 1, wherein said gel tube bottom is removable to allow refilling said gel tube.

4. The gel applicator of claim 2, wherein said gel tube bottom is removable to allow refilling said gel tube.

5. A medical laser gel applicator apparatus, comprising:
a gel tube having an application tip from which gel is applied to a patient, and a removable bottom slidably movable within said tube pushing gel out through said application tip, and wherein said gel tube bottom is removable to allow refilling said gel tube;
a gun having a handle, a chamber into which said gel tube inserts, a plunger with a shaft and a plunger face contactable with said removable bottom, a trigger in electromechanical communication with said plunger, wherein when said trigger is depressed the plunger pushes against said bottom sliding said bottom in said tube, pressuring said gel out of said application tip, and wherein when said trigger is released, said plunger backs off of said bottom, stopping the flow of said gel; and
a stepper motor, a controller in electrical communication with said stepper motor, and gears in mechanical communication with said motor engaging teeth on said plunger shaft.

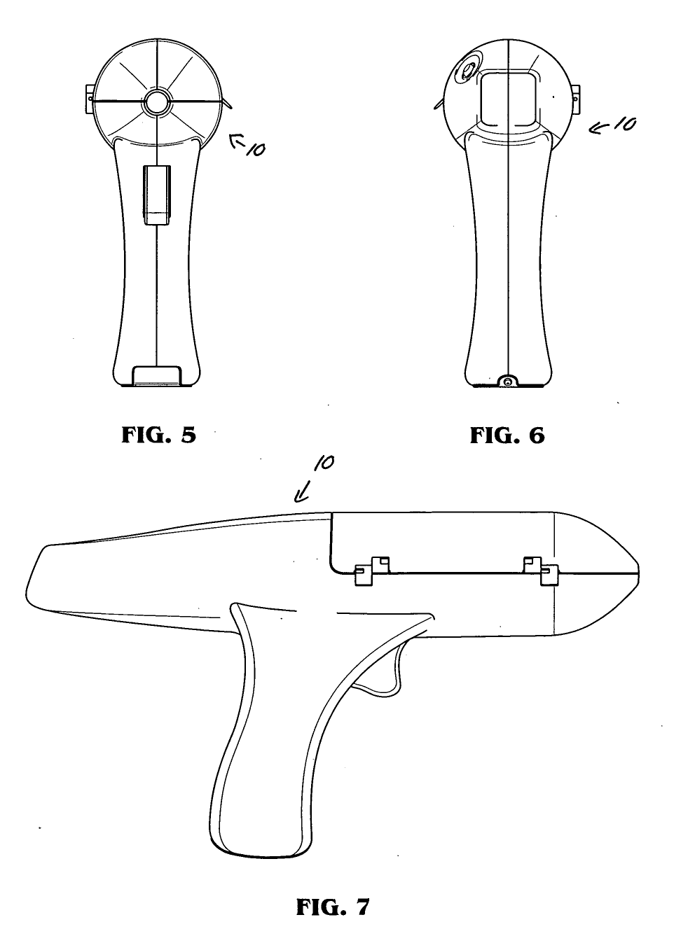

6. The ornamental surgical gel applicator as shown in FIGS. 5-10.
